(19) United States
(12) Patent Application Publication
(10) Pub. No.: US 2023/0355179 A1
(43) Pub. Date: Nov. 9, 2023

(54) PHOTOPLETHYSMOGRAM SYSTEM AND METHOD

(71) Applicant: PRANAQ PTE. LTD., Taipei City (TW)

(72) Inventors: Tzu-Yang Peng, Taipei City (TW); Cheng-Yao Chen, Taipei City (TW); Uei-Ming Jow, San Diego, CA (US)

(73) Assignee: PRANAQ PTE. LTD., Taipei City (TW)

(21) Appl. No.: 18/195,365

(22) Filed: May 9, 2023

Related U.S. Application Data
(60) Provisional application No. 63/339,964, filed on May 9, 2022.

(51) Int. Cl. A61B 5/00; A61B 5/024

(52) U.S. Cl. CPC A61B 5/6843; A61B 5/6826; A61B 5/02427; A61B 2562/046; A61B 2560/0238; A61B 2560/045

(57) ABSTRACT

Photoplethysmogram system and method are designed for monitoring a user's physiological signals. The system includes a PPG device with a light source, a PPG sensor, and at least one verification sensor. The PPG sensor collects light reflected from a user's fingertip after being emitted by the light source, while the verification sensor determines the position of the fingertip and measures the contact pressure of the PPG device to the user's fingertip. Additionally, the system includes an analysis module that is electrically connected to the PPG device, which is responsible for analyzing and processing a plurality of collected data from the device. There is also a display module that is electrically connected to the analysis module, providing a feedback on the PPG device's performance. The analysis module dynamically adjusts the intensity and sampling rate of light emitted by the light source based on the measured contact pressure.

### BACKGROUND OF THE INVENTION

### 1. Field of the Invention

The present invention relates generally to the field of physiological monitoring systems, and more specifically to a photoplethysmogram system and method for monitoring a user's physiological signals by collecting data from a user's fingertip.

### 2. Description of the Prior Art

Photoplethysmography (PPG) is a non-invasive technique for measuring changes in blood volume in tissues using light. PPG devices are widely used in various medical applications, such as heart rate monitoring, blood pressure estimation, and oxygen saturation measurement. These devices typically consist of a light source, such as light-emitting diodes (LEDs), and a photodetector that measures the intensity of light reflected or transmitted through the tissue.

However, PPG devices often suffer from poor signal quality due to various factors, such as the positioning of the device on the user's fingertip, the attachment strength of the device, and the user's skin conditions. Inaccurate measurements may lead to incorrect diagnoses and treatment recommendations. Additionally, PPG devices generally require the user to remain still during the measurement process, which can be inconvenient and uncomfortable for the user.

Thus, there is a need for an improved photoplethysmogram system and method that can provide accurate and reliable physiological monitoring while accommodating various user conditions and ensuring proper device positioning.

As it can be seen, how to solve aforesaid shortcoming becomes an important issue to persons who are skilled in the art.

### SUMMARY OF THE INVENTION

The first objective of the present invention is to provide an improved photoplethysmogram system and method that can provide accurate and reliable physiological monitoring while accommodating various user conditions and ensuring proper device positioning.

To achieve the aforementioned objective and others, a photoplethysmogram system is provided for monitoring a user's physiological signals. The system comprises a PPG device with a light source, a PPG sensor, and at least one verification sensor. The PPG sensor collects light reflected from a user's fingertip after being emitted by the light source, while the verification sensor determines the position of the fingertip and measures the contact pressure of the PPG device to the user's fingertip. Additionally, the system includes an analysis module that is electrically connected to the PPG device, which is responsible for analyzing and processing a plurality of collected data from the device. There is also a display module that is electrically connected to the analysis module, providing a feedback on the PPG device's performance. The analysis module normalizes the collected data based on the measured contact pressure to minimize the variability between different usages. This adjustment aims to enhance the quality of the collected data.

To achieve the aforementioned objective and others, a photoplethysmogram method for monitoring a user's physiological signals is provided and involves several steps. Firstly, a PPG device is attached to the user's fingertip. Secondly, at least one verification sensor is used to check the proper positioning of the PPG device. If the PPG device is not properly positioned, instructions are displayed on a module, guiding the user to reposition the device. Next, data is collected from the PPG device, including PPG signals and fingertip positions. Afterward, an analysis module is utilized to analyze and process the data obtained from the PPG device. Thereafter, the analysis module normalizes the collected data based on the measured contact pressure, which aims to enhance the quality of the collected data. Throughout the monitoring period, both PPG signals and fingertip positions are continuously monitored.

The present invention provides an improved photoplethysmogram-based system and method for monitoring a user's physiological signals, offering enhanced accuracy and reliability by ensuring proper device positioning and accommodating various user conditions.

Other and further features, advantages, and benefits of the invention will become apparent in the following description taken in conjunction with the following drawings. It is to be understood that the foregoing general description and following detailed description are exemplary and explanatory but are not to be restrictive of the invention. The accompanying drawings are incorporated in and constitute a part of this application and, together with the description, serve to explain the principles of the invention in general terms. Like numerals refer to like parts throughout the disclosure.

### BRIEF DESCRIPTION OF THE DRAWINGS

The objects, spirits, and advantages of the preferred embodiments of the present invention will be readily understood by the accompanying drawings and detailed descriptions, wherein:

FIG. 1 shows an overview of the photoplethysmogram system.

FIG. 2A shows a close-up view of the PPG device and its components in one embodiment.

FIG. 2B shows an alternative configuration of the PPG device with the integration of an analysis module and a display module.

FIG. 2C shows multiple pressure sensors strategically positioned around the PPG sensor.

FIG. 3A illustrates a flowchart depicting an embodiment of the photoplethysmogram method for monitoring a user's physiological signals.

FIG. 3B illustrates a flowchart depicting another embodiment of the photoplethysmogram method for monitoring a user's physiological signals.

FIG. 3C illustrates a flowchart depicting an embodiment of the photoplethysmogram method for monitoring a user's physiological signals.

### DETAILED DESCRIPTION OF THE INVENTION

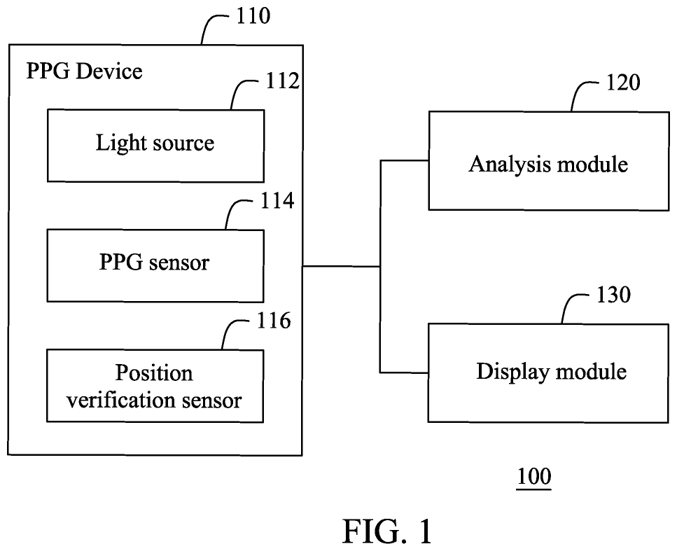

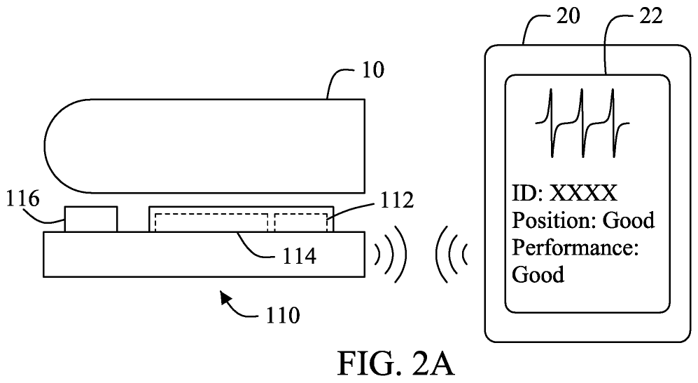

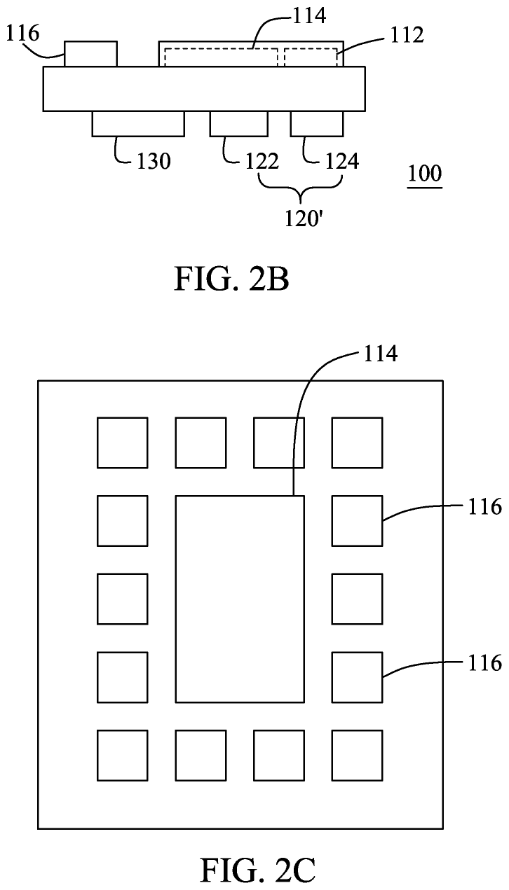

Please refer to FIG. 1 and FIG. 2A. FIG. 1 shows an overview of the photoplethysmogram system and FIG. 2A shows a close-up view of the PPG device 110 and its components in one embodiment. The photoplethysmogram system 100 is designed for monitoring a user's physiological signals and includes a PPG device 110, an analysis module 120, and a display module 130. The PPG device 110 includes a light source 112, a PPG sensor 114, and at least one verification sensor 116. The light source 112, which may include a plurality of light-emitting diodes (LEDs), emits light onto a user's fingertip 10. The PPG sensor 114 collects light reflected from the user's fingertip after being emitted by the light source 112. The verification sensor 116, located next to the PPG sensor 114, is configured to determine the position of the fingertip and measure the contact pressure of the PPG device 110 to the user's fingertip 10. Additionally, it should be noted that the light source 112 and the PPG sensor 114 can be either packaged together as an integrated unit or designed as separate components. In an integrated configuration, the light source 112 and PPG sensor 114 are combined into a single module, which can simplify the overall design and assembly of the PPG device 110. On the other hand, in a separate configuration, the light source 112 and PPG sensor 114 are distinct components, providing more flexibility in terms of placement and design, which may facilitate customization or optimization for specific applications or user needs.

In the embodiment, the analysis module 120 and the display module 130 are installed in an electronic device (e.g., a handheld device such as a smartphone, tablet, or smartwatch), which is communicatively connected to the PPG device 110. The analysis module 120, electrically connected to the PPG device 110, is configured to analyze and process a plurality of collected data from the PPG device 110. The analysis module 120 normalizes the collected data based on the measured contact pressure, enhancing the quality of the collected data. The normalization process is such as dynamically adjusting an intensity of light emitted by the light source 112, dynamically adjusting a sampling rate of light emitted by the light source 112, or mapping the collected data to a pre-trained curve.

The technique of dynamically adjusting the intensity of light emitted by the light source 112 involves continuously adapting the light intensity based on the user's fingertip contact pressure and other factors. By adjusting the light intensity, the photoplethysmogram system 100 ensures optimal illumination conditions for the PPG sensor 114 to collect accurate and reliable data. The technique of dynamically adjusting the sampling rate of light emitted by the light source 112 involves changing the rate at which the PPG sensor 114 collects the reflected light data. A higher sampling rate may reveal more details of the PPG waveform, while a lower sampling rate might be more energy-efficient. The photoplethysmogram system 100 dynamically adjusts the sampling rate based on factors such as contact pressure and collected data quality to strike a balance between accuracy and efficiency. This technique of mapping the collected data to a pre-trained curve involves comparing or aligning the collected data with a pre-established curve or model. The pre-trained curve represents an ideal or reference signal derived from previous data or experiments. By mapping the collected data (i.e. PPG signals) to the pre-trained curve, the photoplethysmogram system 100 can better understand and interpret the PPG signals, which ultimately leads to improved data quality and more accurate monitoring of the user's physiological signals.

In detail, the electronic device (in this embodiment, a smartphone 20) hosts the analysis module 120 and the display module 130 as integral parts of a dedicated application (APP) designed for the photoplethysmogram system 100. The application communicates with the PPG device 110 through wireless technologies, such as Bluetooth or Wi-Fi, to receive and transmit data between the components seamlessly. The analysis module 120, within the application, processes the data collected from the PPG device 110 in real-time, applying various algorithms and filtering techniques to improve data quality and reliability. The algorithms and filtering techniques employed by the analysis module 120 to improve data quality and reliability include signal filtering (e.g., low-pass, high-pass, band-pass, and notch filters), baseline wander removal (e.g., detrending or high-pass filtering), adaptive filtering (e.g., Least Mean Squares and Recursive Least Squares algorithms), artifact removal (e.g., independent component analysis, principal component analysis, or wavelet-based methods), and peak detection (e.g., Pan-Tompkins algorithm or threshold-based methods). The specific choice of techniques depends on the system requirements, desired accuracy, and target application of the photoplethysmogram system 100.

The analysis module 120 dynamically adjusts the intensity and sampling rate of the light source 112 based on the contact pressure measured by the verification sensor 116. The light source 112 comprises multiple LEDs, which can emit light at different wavelengths, such as red, green, and infrared. These LEDs work in conjunction with the PPG sensor 114 to collect high-quality PPG signals. The contact pressure information is transmitted to the smartphone 20 hosting the application, where the analysis module 120 processes the data in real-time. By considering the measured contact pressure, the analysis module 120 can optimize the intensity and sampling rate of the light source 112, ensuring optimal light penetration and minimizing motion artifacts caused by changes in the contact pressure. The adjustment of the light source intensity and sampling rate can be performed using feedback control algorithms, such as proportional-integral-derivative (PID) control or adaptive control techniques. This dynamic adjustment enables the photoplethysmogram system 100 to maintain a high level of data quality and accuracy, even when the user's fingertip positioning or contact pressure changes during the monitoring period.

The display module 130, also part of the application, presents the analyzed data in a user-friendly interface on the smartphone's screen 22, providing the user with real-time feedback and visualizations of their physiological signals. This may include graphical representations of the PPG signal, heart rate, blood oxygen saturation, and other relevant parameters. The display module 130 also provides instructions to the user for repositioning the PPG device 110 if it is not properly positioned, ensuring optimal data quality and accuracy.

Additionally, the analysis module 120 can also be implemented in a cloud-based configuration. In this setup, the PPG device 110 sends the collected data to the electronic device, which then forwards the data to a cloud server for analysis by the analysis module 120. This allows for remote processing and analysis of the collected data, taking advantage of the increased computational power and resources available on the cloud server. By using the cloud-based analysis module 120, the photoplethysmogram system can provide enhanced data processing capabilities and facilitate more advanced and efficient normalization techniques, ultimately improving the overall performance of the photoplethysmogram system.

FIG. 2B shows an alternative configuration of the PPG device 110′, with the integration of an analysis module 120′ and a display module 130′. In the embodiment, the analysis module 120′ includes a microcontroller 122 and a non-transitory storage medium 124. The non-transitory storage medium 124, which is electrically connected to the microcontroller 122, stores an algorithm. The microcontroller 122 uses the algorithm to dynamically adjust the intensity and sampling rate of light emitted by the light source 112 (e.g. LEDs) based on the measured contact pressure, enhancing the quality of the collected data. Here, the algorithm is similar to the one mentioned in the embodiment corresponding to FIG. 2A. In the embodiment, the display module 130′ is such as an OLED (Organic Light Emitting Diode) display, a liquid crystal display, or an E-ink display. Additionally, The display module 130′ can alternatively comprise multiple LEDs (Light Emitting Diodes) of different colors, which can be used as a simple, cost-effective, and energy-efficient way to convey information to the user. These LEDs can be arranged in various configurations, such as in a linear or circular pattern, or as a matrix display. The different colors can represent different levels of positioning accuracy, data quality, or system status. For example, a green LED could indicate that the PPG device is properly positioned on the user's fingertip, while a red LED could signal that the device needs to be repositioned.

Alternatively, a combination of colors, such as a yellow LED, could represent an intermediate state, where the device is not optimally positioned, but still functional. The LEDs could also blink or change colors to communicate different levels of urgency or to guide the user through the repositioning process.

In some embodiments, the analysis module 120, 120′ is configured to calibrate the data recorded based on the contact pressure measured by the verification sensor 116. The calibration process may involve the following steps. First, the analysis module 120, 120′ establishes a baseline for contact pressure when the PPG device 110 is properly positioned on the user's fingertip. This baseline contact pressure value serves as a reference for subsequent measurements. Next, during the monitoring period, the verification sensor 116 continuously measures the contact pressure between the PPG device 110 and the user's fingertip. The analysis module 120, 120′ compares the measured contact pressure with the baseline value to detect any significant deviations. Following this, based on the deviations in contact pressure, the analysis module 120, 120′ adjusts the signal processing parameters accordingly. For example, it may apply different filtering techniques, such as adaptive or notch filters, to remove motion artifacts caused by changes in contact pressure. Then, the analysis module 120, 120′ uses the adjusted signal processing parameters to calibrate the PPG signal, compensating for any variations in contact pressure. This calibration process helps to maintain the accuracy and reliability of the PPG data, even when the contact pressure fluctuates during the monitoring period.

In some embodiment, the verification sensor 116 takes the form of a pressure sensor. As shown in FIG. 2C, there may be multiple pressure sensors strategically positioned around the PPG sensor 114. This arrangement of multiple pressure sensors enhances the system's ability to accurately detect and measure contact pressure between the PPG device 110 and the user's fingertip, ensuring proper positioning and optimal data quality. The use of multiple pressure sensors also allows the system to better account for variations in fingertip size and shape, which can help improve the overall performance and user experience of the photoplethysmogram system 100.

In some embodiment, the verification sensor 116 can alternatively be a capacitive or resistive fingertip sensor. This embodiment provides an alternative approach to the pressure sensors for determining the position of the user's fingertip and measuring the contact pressure between the PPG device 110 and the user's fingertip. the capacitive fingertip sensor detects the changes in capacitance caused by the proximity of the user's fingertip, whereas resistive fingertip sensor measures the changes in resistance due to the applied pressure from the user's fingertip. Both of these sensors can provide valuable information about the position of the fingertip and the contact pressure between the PPG device 110 and the user's fingertip. The capacitive or resistive fingertip sensor can also be configured to authenticate the user by recognizing the unique patterns of their fingerprint, adding an extra layer of security to the photoplethysmogram system 100. This feature can be particularly useful in scenarios where personal data privacy and security are of utmost importance.

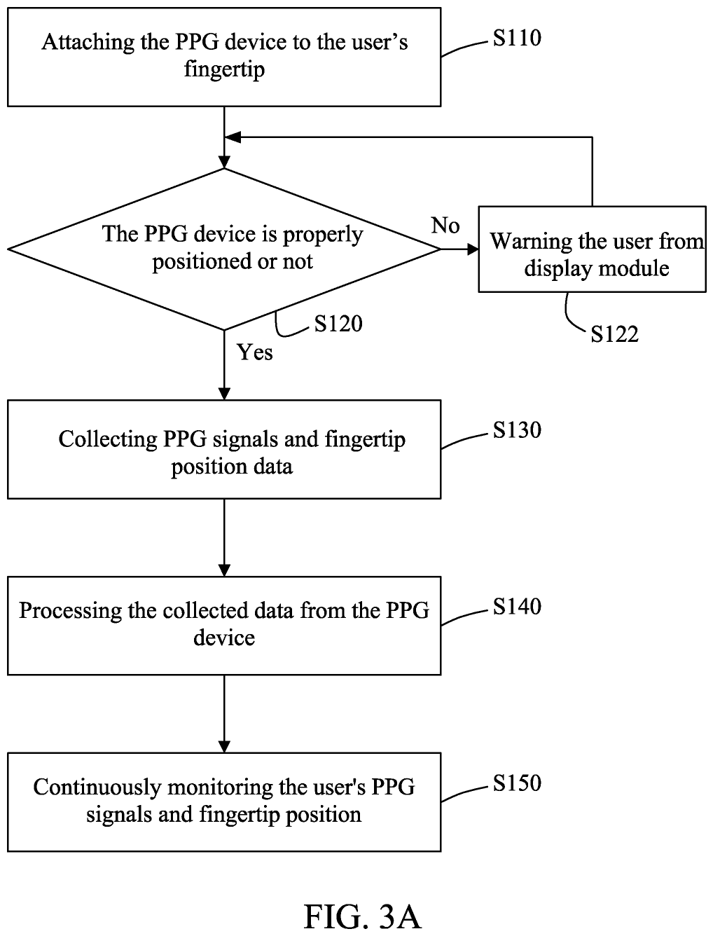

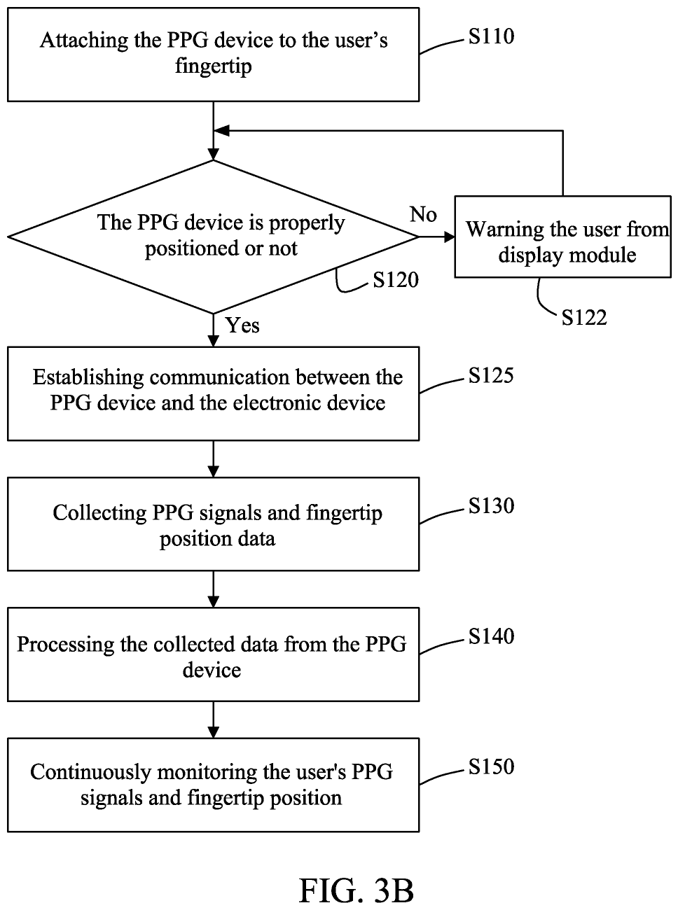

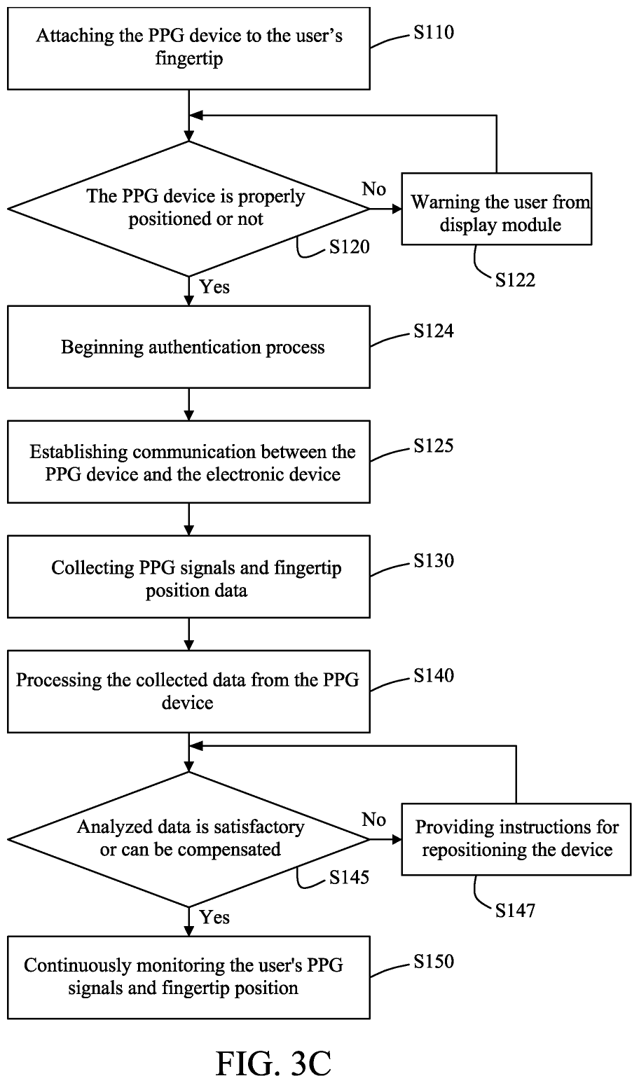

Please refer to FIG. 2A, FIG. 2B, and FIG. 3A. FIG. 3A illustrates a flowchart depicting an embodiment of the photoplethysmogram method for monitoring a user's physiological signals. First, as shown in step S110, the user attaches the PPG device 110 to their fingertip 10, ensuring that the light source 112 and the PPG sensor 114 are in contact with the skin of the user's fingertip 10. Next, as shown in step S120, the verification sensor 116 determines whether the PPG device 110 is properly positioned on the user's fingertip 10. If the PPG device 110 is not correctly positioned, which could be due to the PPG device 110 being fastened too loosely, resulting in insufficient or unstable contact area, the display module 130 provides instructions to the user for repositioning the PPG device 110 (as shown in step S122). The verification sensor 116 works to determine whether the PPG device 110 is properly positioned on the user's fingertip by measuring the contact pressure between the device and the user's skin. In some embodiments, the verification sensor 116 can be a pressure sensor, a capacitive sensor, or a resistive fingertip sensor. These sensors can detect the changes in pressure, capacitance, or resistance, respectively, as a result of the device's contact with the fingertip. When the PPG device 110 is correctly positioned, the contact pressure or the changes in capacitance or resistance will be within a predefined range, indicating proper alignment of the light source 112 and the PPG sensor 114 with the user's blood vessels.

Once the PPG device 110 is properly positioned, it begins collecting PPG signals and fingertip position data, as shown in step S130. The light source 112 emits light into the user's fingertip, and the PPG sensor 114 measures the changes in light intensity resulting from blood flow changes. Following data collection, the analysis module 120 processes the collected data from the PPG device 110, as shown in step S140, applying various algorithms and filtering techniques to improve data quality and reliability. It also dynamically adjusts the intensity and sampling rate of the light source 112 based on the contact pressure measured by the verification sensor 116, enhancing the quality of the collected data. Then, as shown in step S150, the photoplethysmogram system 100 continuously monitors the user's PPG signals and fingertip position throughout the monitoring period, providing real-time feedback on the user's physiological signals.

Please refer to FIG. 2A and FIG. 3B. FIG. 3B illustrates a flowchart depicting another embodiment of the photoplethysmogram method for monitoring a user's physiological signals. First, as shown in step S110, the user attaches the PPG device 110 to their fingertip 10, ensuring that the light source 112 and the PPG sensor 114 are in contact with the skin of the user's fingertip 10. Next, as shown in step S120, the verification sensor 116 determines whether the PPG device 110 is properly positioned on the user's fingertip 10. If the PPG device 110 is not correctly positioned, the display module 130 provides instructions to the user for repositioning the PPG device 110 (step S122).

Once the PPG device 110 is properly positioned, as shown in step S125, the communication between the PPG device 110 and the electronic device (such as the smartphone 20 shown in FIG. 2A) hosting the analysis module 120 and the display module 130 is established. The communication can be accomplished through various means, including Bluetooth, Wi-Fi, or a wired connection.

After establishing communication, the PPG device 110 begins to collect data, including PPG signals and fingertip positions, as shown in step S130. The analysis module 120 then processes the collected data from the PPG device in real-time, applying various algorithms and filtering techniques to improve data quality and reliability (as shown in step S140). In the step S140, the analysis module 120 dynamically adjusts the intensity and sampling rate of the light source 112 based on the contact pressure measured by the verification sensor 116 to enhance the quality of the collected data.

Then, as shown in step S150, the photoplethysmogram system 100 continuously monitors the PPG signals and fingertip position during the monitoring period, allowing for ongoing analysis and adjustments as needed.

Please refer to FIG. 2A and FIG. 3C. FIG. 3C illustrates a flowchart depicting yet another embodiment of the photoplethysmogram method for monitoring a user's physiological signals. In this embodiment, the photoplethysmogram method begins with attaching the PPG device 110 to the user's fingertip, as shown as in step S110. Following this step, the verification sensor 116 determines whether the PPG device 110 is properly positioned on the user's fingertip, as shown as in step S120. If the PPG device 110 is not correctly positioned, the display module 130 provides instructions to the user for repositioning the PPG device 110. In the embodiment, the verification sensor 116 is a capacitive or resistive fingertip sensor.

Once the PPG device 110 is properly positioned, the user authentication process begins by utilizing the capacitive or resistive fingertip sensor (i.e. the verification sensor 116), as shown as in step S124. This authentication step, i.e. the step S124, is crucial for ensuring that the collected data is associated with the correct user and for maintaining the security of personal health information. By authenticating the user before establishing communication with the electronic device, the system can provide a more secure and accurate monitoring experience. If the authentication is successful, the system proceeds to the next step; if the authentication fails, the process is terminated.

After successful authentication, communication is established between the PPG device 110 and the electronic device, as shown as in step S125. The method then proceeds with collecting data from the PPG device 110, including PPG signals and fingertip position, as shown as in step S130, and analyzing and processing the collected data using the analysis module as shown as in step S140.

The next step, i.e. step 145, is to determine if the quality of the analyzed data is satisfactory or can be compensated. This step is particularly important during sleep monitoring, as it aims to minimize the disruption of the user's sleep. If the data quality is not satisfactory and cannot be compensated, the display module 130 provides instructions for repositioning the device. This way, the user is only alerted when absolutely necessary, preventing unnecessary interruptions during sleep. Then, as shown as in step S150, the method continues with the continuous monitoring of the PPG signals and fingertip position during the monitoring period.

Although the invention has been disclosed and illustrated with reference to particular embodiments, the principles involved are susceptible for use in numerous other embodiments that will be apparent to persons skilled in the art. This invention is, therefore, to be limited only as indicated by the scope of the appended claims.

What is claimed is:

1. A photoplethysmogram system for monitoring a user's physiological signals, comprising:
a PPG device comprising:
a light source;
a PPG sensor configured to collect light reflected from a user's fingertip after being emitted by the light source; and
at least one verification sensor, located next to the PPG sensor, the verification sensor configured to determine a position of the fingertip and measure a contact pressure of the PPG device to the user's fingertip;
an analysis module, electrically connected to the PPG device, the analysis module configured to analyze and process a plurality of collected data from the PPG device;
a display module electrically connected to the analysis module and configured to provide a feedback on a performance of the PPG device;
wherein the analysis module normalizes the collected data based on the measured contact pressure and enhance the quality of the collected data.

2. The photoplethysmogram system of claim 1, wherein the verification sensor is a pressure sensor.

3. The photoplethysmogram system of claim 1, wherein there are multiple pressure sensors.

4. The photoplethysmogram system of claim 1, wherein the pressure sensors are surrounding the PPG sensor.

5. The photoplethysmogram system of claim 1, wherein the verification sensor is a capacitive or resistive fingertip sensor, and the fingertip sensor is configured to authenticate the user and determine the position of the fingertip.

6. The photoplethysmogram system of claim 1, wherein the analysis module comprising a microcontroller and a non-transitory storage medium, the non-transitory storage medium is electrically connected to the microcontroller and stores an algorithm, the microcontroller and the non-transitory storage medium as well as the display module are integrated within the PPG device, and the microcontroller uses the algorithm to dynamically adjust the intensity and sampling rate of light emitted by the light source based on the measured contact pressure and enhance the quality of the collected data.

7. The photoplethysmogram system of claim 1, wherein the analysis module and the display module are installed in an electronic device and the electronic device is communicatively connected to the PPG device.

8. The photoplethysmogram system of claim 1, wherein the electronic device is a handheld device.

9. The photoplethysmogram system of claim 1, wherein the analysis module is configured to calibrate the data recorded based on the contact pressure measured by the pressure sensor.

10. The photoplethysmogram system of claim 1, wherein the light source comprises a plurality of light emitting diodes.

11. The photoplethysmogram system of claim 1, wherein the analysis module normalizes the collected data by dynamically adjusting an intensity of light emitted by the light source.

12. The photoplethysmogram system of claim 1, wherein the analysis module normalizes the collected data by dynamically adjusting a sampling rate of light emitted by the light source.

13. The photoplethysmogram system of claim 1, wherein the analysis module normalizes the collected data by mapping the collected data to a pre-trained curve.

14. A photoplethysmogram method of monitoring a user's physiological signals comprising:
(a) attaching a PPG device to a user's fingertip;
(b) using at least one verification sensor to determine if the PPG device is positioned properly and using a display module to provide instructions to the user for repositioning the device if the PPG device is not properly positioned;
(c) collecting data from the PPG device, including PPG signals and fingertip positions;
(d) using an analysis module to analyze and process the collected data from the PPG device; and
(e) continuously monitoring the PPG signals and the fingertip position during the monitoring period;
wherein the analysis module normalizes the collected data based on the measured contact pressure and enhances the quality of the collected data.

15. The photoplethysmogram method of claim 14, wherein the verification sensor is a pressure sensor.

16. The photoplethysmogram method of claim 14, wherein the analysis module and the display module are installed in an electronic device, and the photoplethysmogram method further comprises a step of establishing communication between the PPG device and the electronic device between the step (b) and the step (c).

17. The photoplethysmogram method of claim 14, further comprising a step of: if the analysis module determines that the quality of the analyzed data is not satisfactory and cannot be compensated, using the display module to provide instructions to the user for repositioning the device between step (d) and step (e).

18. The photoplethysmogram method of claim 14, wherein the verification sensor is a capacitive or resistive fingertip sensor, and the fingertip sensor is configured to authenticate the user and determine the position of the fingertip.

19. The photoplethysmogram method of claim 14, wherein the analysis module normalizes the collected data by dynamically adjusting an intensity of light emitted by the light source.

20. The photoplethysmogram method of claim 14, wherein the analysis module normalizes the collected data by dynamically adjusting a sampling rate of light emitted by the light source.
